(19) United States
(12) Patent Application Publication
(10) Pub. No.: US 2019/0029593 A1
(43) Pub. Date: Jan. 31, 2019

(54) EARBUD WITH PHYSIOLOGICAL SENSOR AND STABILIZING ELEMENT

(71) Applicant: LifeBEAM Technologies Ltd., Tel-Aviv (IL)

(72) Inventors: Zvi ORRON, Tel-Aviv (IL); Tali ZICHRONY, Kochav Yair (IL); Jonathan APRASOFF, Kfar-Saba (IL)

(21) Appl. No.: 16/081,059

(22) PCT Filed: Apr. 13, 2017
(86) PCT No.: PCT/IL2017/050451

§ 371 Date: Aug. 30, 2018

Related U.S. Application Data
(60) Provisional application No. 62/323,727, filed on Apr. 17, 2016.

(51) Int. Cl. A61B 5/00; H04R 1/10; A61B 5/024

(52) U.S. Cl. CPC A61B 5/683; H04R 1/105; H04R 1/1016; H04R 1/1041; A61B 2562/0219; A61B 5/02433; A61B 5/02438; A61B 5/0004; H04R 2420/07; A61B 5/6817

(57) ABSTRACT

An earbud for use with, for example, a media player or a smartphone is provided. The earbud includes a housing enclosing a speaker and an optical sensor positioned on the housing such that the optical sensor contacts a surface of tragus when the earbud is positioned in an ear with the speaker directed into an ear canal. The earbud further includes a stabilizing element attachable to the housing and configured for contacting a posterior wall of a cavum or incisura of the ear when the earbud is positioned in the ear with the speaker directed into the ear canal.

### FIELD AND BACKGROUND OF THE INVENTION

The present invention relates to an earbud having a stabilizing element configured for stabilizing an earbud-mounted physiological sensor against tissue of an ear and, more particularly, to an earbud having a shark fin-shaped element for biasing an optical physiological sensor positioned on the earbud against a tragus region of an ear.

Most media players and smartphones sold today ship with in-ear earphones (also referred to as earbuds) which are typically used for listening to audio content.

While current earbud design provides sufficient in-ear stability when a user is stationary, earbuds can occasionally fall out of the ears of a user during vigorous activity such as running.

In order to address this problem, earbud manufacturers have introduced stabilizing elements such as fins that stabilize the earbud against external ear regions or ear-tips which stabilize the earbuds by increasing friction between the earbud and ear canal.

Such stabilizing elements were designed in order to stabilize the entire earbud within the ear in order to maintain audio coupling between the earbud speaker and ear canal.

In recent years, earbuds have evolved to include physiological sensors such as PPG heart rate sensors for measuring for example, a user's heart rate for fitness and health purposes.

Although prior art stabilization elements such as those described above are generally effective in maintaining the earbud speaker within the user's ear canal, the present inventors uncovered that such stabilization elements are insufficient for stabilizing earbud-mounted optical sensors such as PPG sensors which are very sensitive to user movements as well as being suboptimal in as far as correctly positioning the sensor at the desired tissue region.

There is thus a need for, and it would be highly advantageous to have an earbud which is specifically configured to maximize the stability of optical coupling between an earbud optical sensor and ear tissue during user movement.

### SUMMARY OF THE INVENTION

According to one aspect of the present invention there is provided an earbud comprising: (a) a housing enclosing a speaker; (b) an optical sensor positioned on the housing such that the optical sensor contacts a surface of tragus when the earbud is positioned in an ear with the speaker directed into an ear canal; and (c) a stabilizing element attachable to the housing and being configured for contacting a posterior wall of a cavum or incisura of the ear when the earbud is positioned in the ear with the speaker directed into the ear canal.

According to further features in preferred embodiments of the invention described below, the stabilizing element is attachable to the housing at one of several preset rotational positions around the housing.

According to still further features in the described preferred embodiments the optical sensor is a photoplethysmography (PPG) sensor.

According to still further features in the described preferred embodiments the earbud further comprising an accelerometer positioned in or on the housing.

According to still further features in the described preferred embodiments the housing includes a plurality of circumferentially spaced engagement elements for engaging the stabilizing element at one of the several preset rotational positions around the housing.

According to still further features in the described preferred embodiments the stabilizing element is shaped as a shark fin with a base of the shark fin surrounding a portion of a circumference of the housing.

According to still further features in the described preferred embodiments a portion of a front curve extending from a base of the shark fin is configured for contacting the posterior wall of a cavum or incisura of the ear when the earbud is positioned in the ear with the speaker directed into the ear canal.

According to still further features in the described preferred embodiments the front curve has a radius of curvature of 12-15 mm.

According to still further features in the described preferred embodiments a tip of the shark fin contacts the superior Cymba or Antihelix region of the ear when the earbud is positioned in the ear with the speaker directed into the ear canal.

According to still further features in the described preferred embodiments the earbud further comprising an eartip attachable to the housing and being positionable within the ear canal.

According to still further features in the described preferred embodiments n the stabilizing fin and the eartip are removably attached to the housing.

According to still further features in the described preferred embodiments the stabilizing fin and the earpiece are connected.

According to another aspect of the present invention there is provided an earbud comprising: (a) a housing enclosing a speaker, the housing having a flat face being for contacting a surface of a tragus when the ear bud is positioned in an ear with the speaker directed into an ear canal at a specific rotational angle with respect to an axis extending out of the ear canal; and (b) an optical sensor positioned on the flat face of the housing.

According to still further features in the described preferred embodiments the earbud further comprising a stabilizing element attachable to the housing and being configured for contacting a posterior wall of a cavum or incisura of the ear when the earbud is positioned in the ear with the speaker directed into the ear canal.

According to still further features in the described preferred embodiments the stabilizing element is attachable to the housing at one of several preset rotational positions around the housing.

According to still further features in the described preferred embodiments n the optical sensor is a photoplethysmography (PPG) sensor.

According to still further features in the described preferred embodiments the earbud further comprising an accelerometer positioned in or on the housing.

According to still further features in the described preferred embodiments the earbud further comprising a digital signal processor (DSP) within the housing.

According to still further features in the described preferred embodiments the stabilizing element is shaped as a shark fin with a base of the shark fin surrounding a portion of a circumference of the housing.

According to still further features in the described preferred embodiments a portion of a front curve extending from a base of the shark fin is configured for contacting the posterior wall of a cavum or incisura of the ear when the earbud is positioned in the ear with the speaker directed into the ear canal.

According to still further features in the described preferred embodiments n the front curve has a radius of curvature of 12-15 mm.

According to still further features in the described preferred embodiments a tip of the shark fin contacts the superior Cymba or Antihelix region of the ear when the earbud is positioned in the ear with the speaker directed into the ear canal.

The present invention successfully addresses the shortcomings of the presently known configurations by providing an earbud stabilizing element or earbud housing configuration for optimizing the position of an optical sensor of the earbud and stabilizing the optical coupling between the sensor and ear tissue.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a prior art image showing the various anatomical regions of a human ear.

FIGS. 2a-b illustrate a prior art earbud with a typical shark fin-shaped stabilizing element (FIG. 2a) and as positioned in a human ear (FIG. 2b).

FIGS. 3a-c illustrate an embodiment of the present earbud which includes a shark fin stabilizing element (FIG. 3a) configured for stabilizing optical coupling between an optical sensor and tragus tissue of an ear (FIG. 3b-c).

FIGS. 4a-b illustrate a rotational position attachment mechanism of the present stabilizing fin (FIG. 4a) and the positioning of the stabilizing element with respect to the sensor face (FIG. 4b).

FIGS. 5a-b schematically illustrates the prior art shark fin element superimposed over the shark fin element of the present invention (FIG. 5a) and as positioned in a human ear (FIG. 5b).

FIGS. 6a-b illustrate another embodiment of the present earbud in which the sensor face of the housing is configured for aligning and stabilizing optical coupling between the sensor face and tragus tissue.

### DESCRIPTION OF THE PREFERRED EMBODIMENTS

The present invention is of an earbud which is designed for maximizing stability between an earbud sensor and ear tissue. Specifically, the present invention can be used to stabilize optical coupling between a PPG sensor of an earbud and ear tissue.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Prior art earbud stabilizing elements are designed for stabilizing the ear-tip portion of an earbud within the ear canal in order to maintain audio coupling between the speaker (surrounded by the ear tip) and the ear canal.

Stabilizing elements such as shark fins are designed to engage one or more anatomical regions of the ear in order to provide such stability.

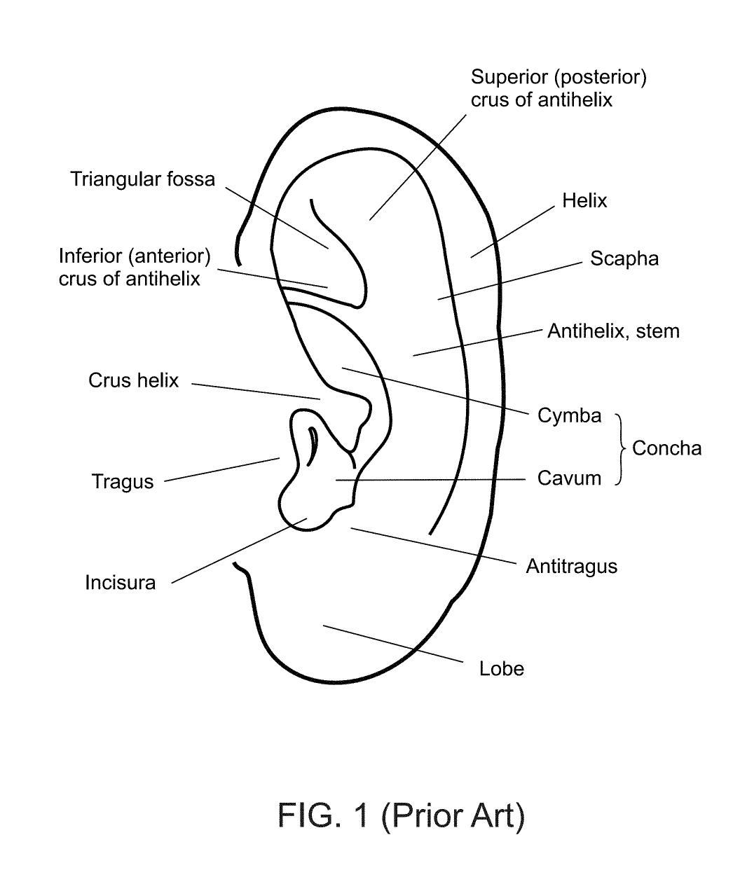

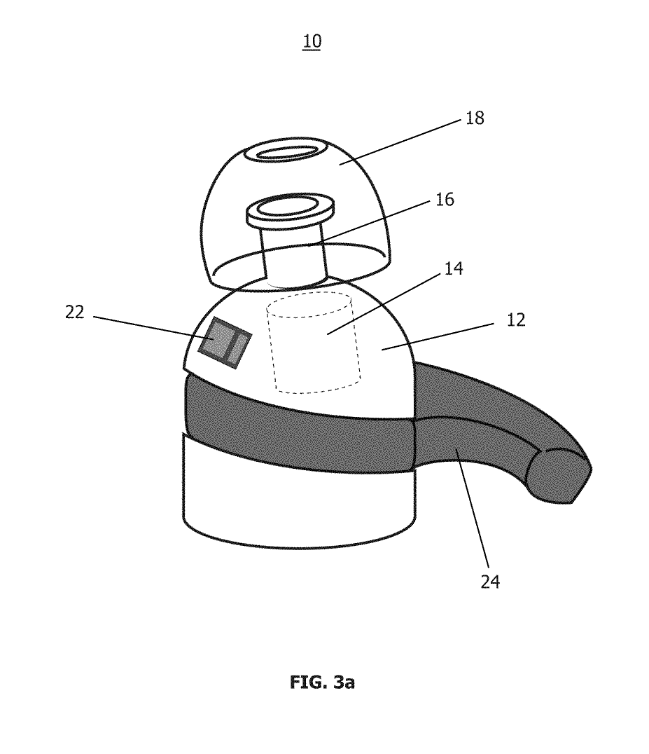

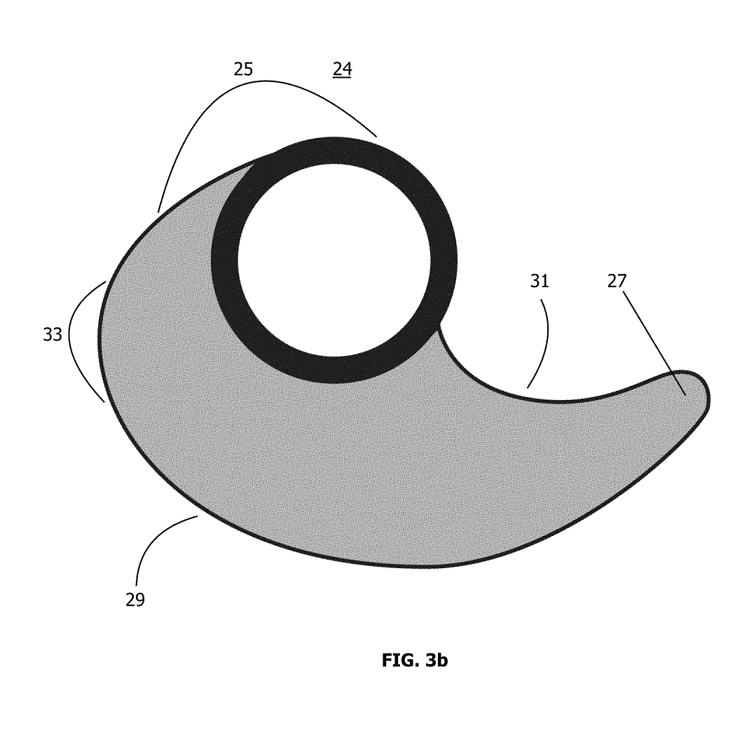

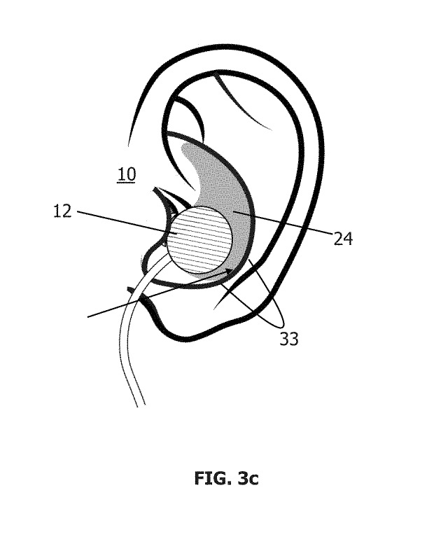

For purposes of better understanding the present invention, as illustrated in FIGS. 3a-6b of the drawings, reference is first made to FIG. 1 which illustrates the various anatomical regions of an external ear.

FIG. 1 illustrates the anatomical regions of a human external ear (also known as the auricle or pinna). The external ear includes the antihelix, a Y-shaped curved cartilaginous ridge arising from the antitragus and separating the concha, triangular fossa, and scapha. The antihelix represents a folding of the conchal cartilage and it usually has similar prominence to a well-developed helix. The stem (the part below the bifurcation) of the normal antihelix is gently curved and branches about two thirds of the way along its course to form the broad fold of the superior (posterior) antihelical crus, and the more sharply folded inferior (anterior) crus. The inferior and superior crura of the antihelix can vary both in volume and degree of folding. The antitragus is the anterosuperior cartilaginous protrusion lying between the incisura and the origin of the antihelix. The anterosuperior margin of the antitragus forms the posterior wall of the incisura. The concha is the fossa bounded by the tragus, incisura, antitragus, antihelix, inferior crus of the antihelix, and root of the helix, into which opens the external auditory canal. It is usually bisected by the crus helix into the cymba superiorly and cavum inferiorly. The Frankfurt Horizontal is a plane connecting the lowest point on the lower margin of each orbit and highest point on the upper margin of the external auditory meatus. The Frankfurt horizontal or Frankfurt plane is used as the general horizontal plane of the head and as reference point for other planes and structures. The helix is the outer rim of the ear that extends from the superior insertion of the ear on the scalp (root) to the termination of the cartilage at the earlobe. The helix can be divided into three approximate parts: the ascending helix, which extends vertically from the root; the superior helix, which begins at the top of the ascending portion, extends horizontally and curves posteriorly to the site of Darwin tubercle (vide infra); the descending helix (sometimes called posterior), which begins inferior to Darwin tubercle and extends to the superior border of the earlobe. The lower portion of the posterior part is often non-cartilaginous. The border of the helix usually forms a rolled rim but the helix is highly variable in shape. The crus of the helix is the continuation of the anteroinferior ascending helix, which extends in a posteroinferior direction into the cavity of the concha above the external auditory meatus. The average crus helix extends about one half to two thirds the distance across the concha. The lobe is the soft, fleshy, inferior part of the pinna. It is bounded on its posterosuperior border by the end of the descending helix, on the anterosuperior border by the inferior border of the antitragus and superiorly by the incisura. The earlobe is highly variable in size and in the degree of attachment of the anteroinferior portion to the face. The scapha is the groove between the helix and the antihelix while the tragus is a posterior, slightly inferior, protrusion of skin-covered cartilage, anterior to the auditory meatus. The inferoposterior margin of the tragus forms the anterior wall of the incisura. The triangular Fossa is the concavity bounded by the superior and inferior crura of the antihelix and the ascending portion of the helix. The scapha is the groove between the helix and the antihelix and the tragus is a posterior, slightly inferior, protrusion of skin-covered cartilage, anterior to the auditory meatus. The inferoposterior margin of the tragus forms the anterior wall of the incisura. The triangular Fossa is the concavity bounded by the superior and inferior crura of the antihelix and the ascending portion of the helix.

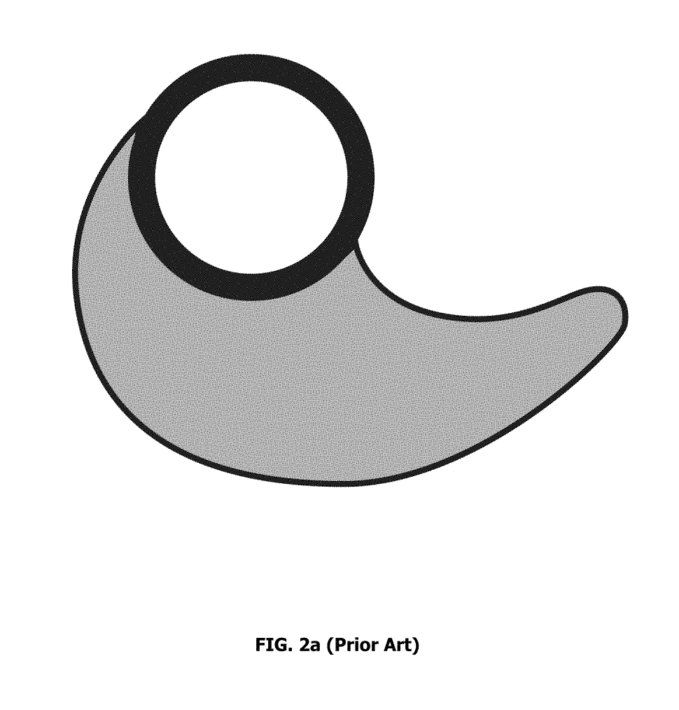

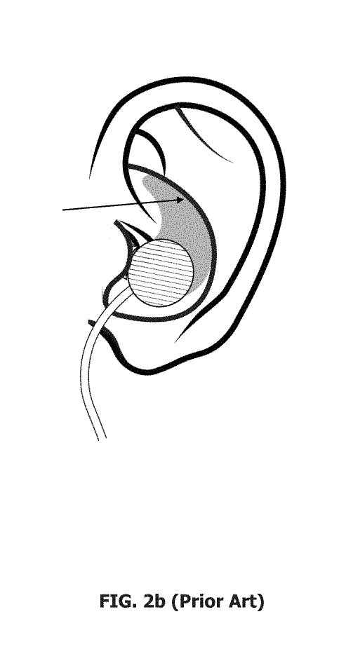

FIGS. 2a-b illustrate a prior art earbud having a typical shark fin-shaped stabilizing element shown out of the ear in FIG. 2a and in the ear in FIG. 2b. When positioned in a human ear (FIG. 2b), the stabilizing element is designed for contacting the superior Cymba or Antihelix region (arrow) thus pushing the earbud towards the ear-canal, against the exterior part of the Antihelix. This force ensures the earbud's ear-tip maintain acoustic sealing with the ear canal even during vigorous exercise.

While such stabilization is sufficient for stabilizing the ear-tip within the ear canal (the intended purpose of prior art stabilizing elements), it does not provide the stability necessary for maintaining optical coupling between an optical sensor and tragus tissue since. The optical sensor is sensitive to even the slightest relative movement against the skin. Such movement does not affect the acoustic seal due to the flexible nature of the silicone eartip. Since the optical sensor is hard-coupled to the earbud housing, there is a need for increased stability.

Thus, according to one aspect of the present invention there is provided an earbud for use in a headset. The headset can be wired to an audio or data port of a mobile device (e.g. media player such as an MP3 player or a smartphone) or it can wirelessly connect thereto via BlueTooth or the like. Although the following describes a single earbud, it is to be understood that in a headset having a pair of earbuds, both earbuds can be configured as described below.

The earbud includes a housing enclosing a speaker such as Chia-Ping CEHR-057NWG or CE-0B-098NTDWG. The earbud can further include an ear-tip attached to the housing and surrounding the speaker-out opening. The housing can be shaped with a volume of 7-12 cm³. The housing can be molded from a polymer such as polycarbonate. The ear-tip can be cone-shaped and fabricated from silicone.

The housing can further include printed circuit boards including analog signal-conditioning elements, accelerometers, capacitance based touch sensors, LEDs, and gyroscopes.

The earbud further includes an optical sensor positioned on the housing at a region that contacts a surface of a tragus when the earbud is positioned in an ear with the speaker directed into an ear canal.

The optical sensor can be a photoplethysmography (PPG) sensor having at least one photodiode for generating light at a wavelength of 530 nm and at least one photodetector for detecting light reflected from the tissue. The sensor can also include at least one photodiode for emitting infra-red light at a wavelength of 880 nm. The sensor can further include an analog front end element for signal conditioning and amplification. The sensor can also include an analog-to-digital sampling element for recording the signals. The sensor can further include a digital interface allowing a micro-controller to read the recorded signals and configure the sensor's parameters.

The optical sensor is positioned on the housing with the sensor face flush with or slightly protruding from the housing surface.

The earbud further includes a stabilizing element attachable to the housing and configured for contacting a posterior-inferior wall of a cavum or incisura of the ear when the earbud is positioned in the ear with the speaker directed into the ear canal.

This contact area is slightly further from the ear canal than the surrounding surfaces of the Antihelix. Thus a rotational locking mechanism is achieved. Furthermore, such a stabilizing fin configuration ensures that the user will experience some discomfort if the earbud is not positioned correctly.

FIG. 3a illustrates one embodiment of the present earbud which is referred to herein as earbud 10.

Earbud 10 includes a housing 12 surrounding a speaker 14. The housing includes a nozzle portion 16 having an opening 18 for communicating audio to an ear canal opening. Nozzle portion 16 is surrounded by an ear-tip 18 which is fabricated from an elastic material (e.g. silicone Shore A 50). Ear-tip 18 is stretched over nozzle portion 16 and is attached thereto via friction. Ear-tip 18 can be replaced and can come in various sizes and geometries to fit various ear canal sizes and geometries.

Earbud 10 also includes optical sensor 22 having an optical face flush with or protruding from a region 24 of housing 12. Sensor 22 includes at least one optical emitter (photodiode) and at least one optical detector (photodetector). Sensor 22 is wired to a printed circuit board positioned within housing 12. Housing 12 can also enclose buttons, accelerometers and analog circuitry (not shown).

Earbud 10 also includes stabilizing element 24 (shown separately in FIG. 3b) which in the embodiment shown is shaped as a shark fin having a base 25 and a tip 27 connected via an outer curve 29 and an inner curve 31 (FIG. 3b). Alternative shapes for stabilizing element 24 include a single flexible cylinder, or a closed loop spanning the full length of the anti-tragus. Stabilizing element 24 is fabricated from an elastic material such as silicone (Shore A 60). Stabilizing fin can be solid or perforated to reduce mass.

Stabilizing element 24 is removably attached to housing 12 via one of several mechanisms and can be oriented around a circumference of housing 12 at one of several preset positions.

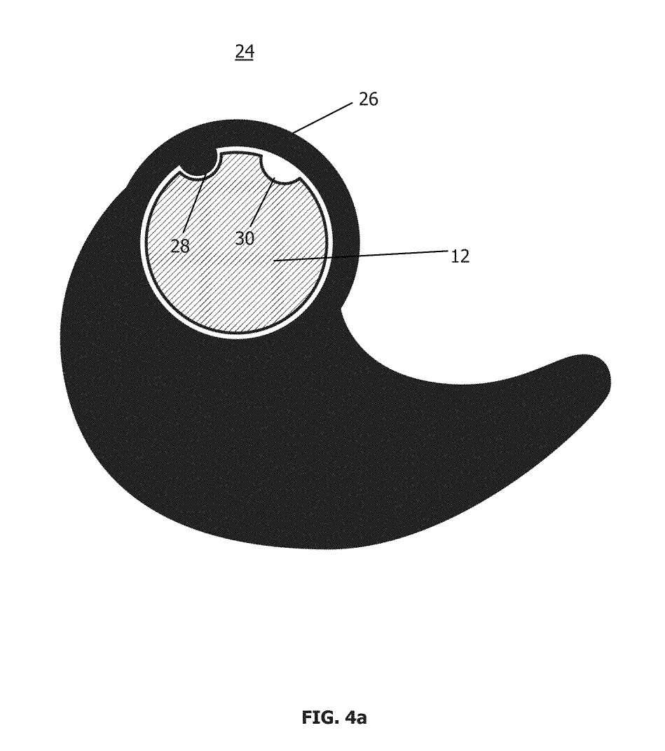

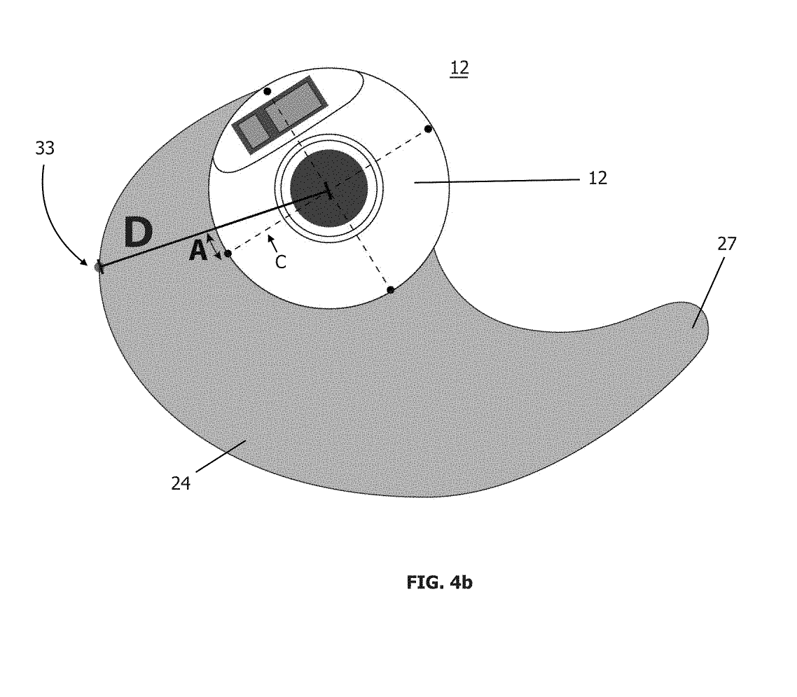

In the embodiment shown in FIG. 4a, stabilizing element 24 includes an elastic ring portion 26 which can be stretched over a circumference of housing 12. A tooth 28 positioned within ring portion 26 can mate with one of several indents 30 positioned around housing 12. This enables custom matching of earbud 10 and stabilizing element 24 to various different ear anatomies.

Since the position of stabilizing element 24 with respect to housing can impact the quality of optical coupling between sensor 22 and the tragus, a user can test several rotational positions against sensor reading (as processed by a mobile device into HR) when vigorously moving (e.g. running) to determine the best possible position for stabilizing element 24. The sensor readings can be automatically processed and compared using a mobile device application, suggesting the optimal rotational position for the user.

When earbud 10 is positioned in an ear of a user (FIG. 3c), stabilizing fin 24 contacts and applies pressure to a posterior wall of a cavum or incisura of the ear (arrow). Since the distance of this anatomical region from the ear-canal is greater than the distances of contact to other anatomical regions, stabilizing element 24 locks earbud 10 from rotating about the axis of the speaker nozzle, forcing the optical sensor to remain biased against the Tragus.

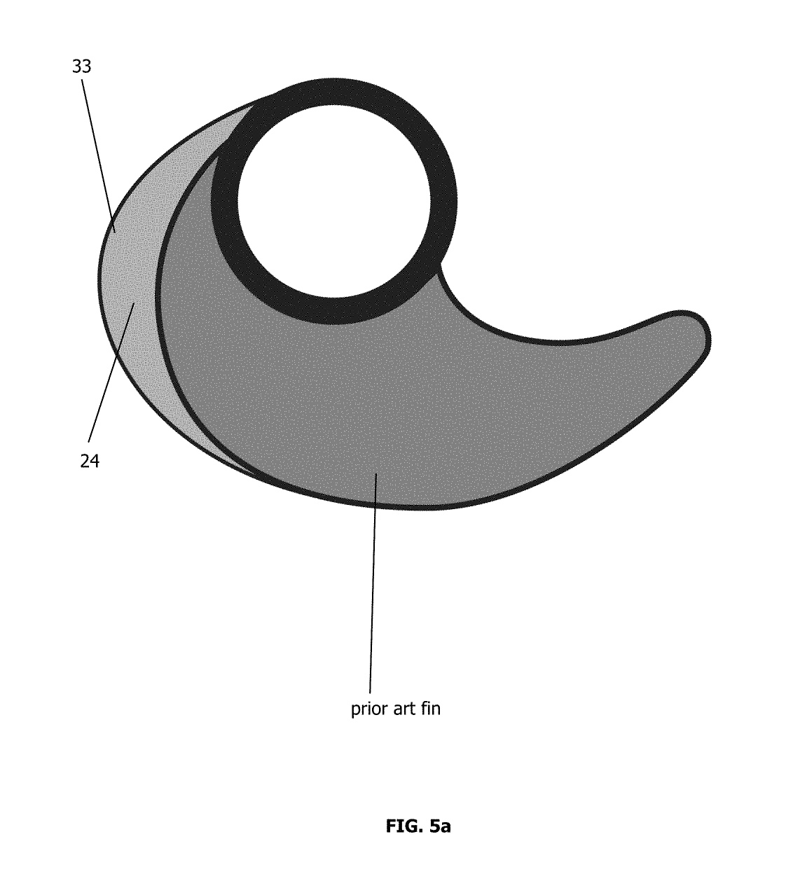

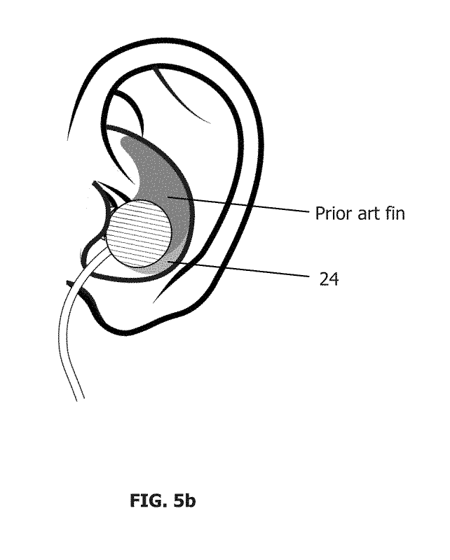

Unlike prior art stabilizing elements, such as the shark fin shown in FIGS. 2a-b which contacts the superior Cymba or Antihelix region of the ear via the shark fin tip, the present stabilizing element contacts the posterior wall of a cavum or incisura of the ear via a lower sector 33 of outer curve 29 (FIGS. 3b-c). As is shown in FIGS. 5a-b, this sector of the present stabilizing element is pronounced (with respect to the prior art stabilizing element) in order to facilitate contact with the posterior wall of a cavum or incisura and apply a force thereto when earbud 10 is positioned within the ear.

As is shown in FIG. 4b, when attached to housing 12, the apex of sector 33 of stabilizing element (at point of contact with ear, on a medium sized stabilizing element) is at a distance (D) of 13.5 mm from the central axis of the earbud housing, at an angle of about 10 degrees from a line (C) that is parallel to the longitudinal axis of sensor 22 and running through the central axis of the earbud.

Stabilizing element 24 can also be shaped to include additional regions of contact for providing overall stability to the earbud. For example, fin tip 27 of a shark fin embodiment of stabilizing element 24 (FIGS. 3b, 4b) can contact the superior Cymba or Antihelix region of the ear much like prior art stabilizing elements.

Stabilizing element 24 and ear-tip 20 can be co-molded as a single unit attachable to housing 12 at one of several orientations (as described above). A single unit stabilizing element and ear-tip is advantageous in that production cost is lower and the product design can be more uniform with less partitions and material breaks.

Stabilizing element 24 and ear-tip 20 or a single unit combination of both can be provided in several sized/geometries as a kit. A user can then try out several sizes/geometries for comfort and functionality of the optical sensor (e.g. the calibration described above).

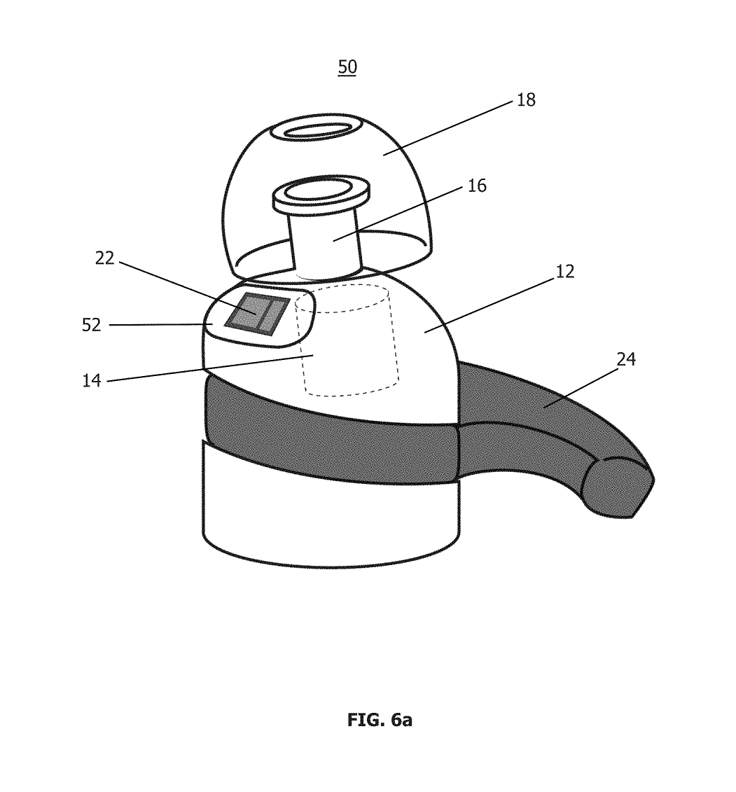

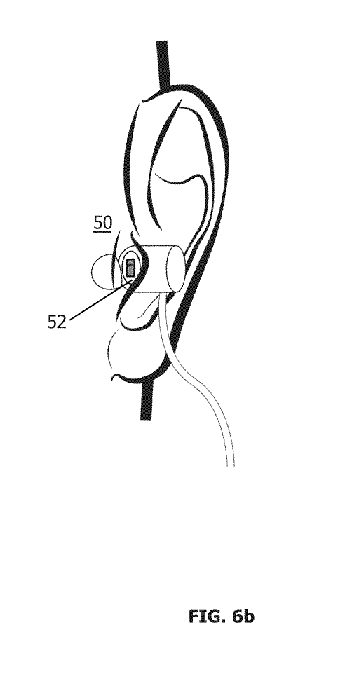

According to another embodiment of the present invention (shown in FIGS. 6a-b) there is provided an earbud for use in a headset. The earbud includes a housing enclosing a speaker, the housing having a flat face for contacting a surface of a tragus when the ear bud is positioned in an ear with the speaker directed into an ear canal at a specific rotational angle with respect to an axis extending out of the ear canal. The earbud further includes an optical sensor positioned on the flat face of the housing.

FIG. 5a illustrates an earbud 50 having a housing configured for stabilizing optical coupling between an optical sensor and a tragus region of an ear.

Earbud 50 is similar in construction to earbud 10 however, it provides stability via geometry and not an attachable stabilizing element. A stabilizing element 24 can optionally be utilized with earbud 50 for added stability.

Housing 12 of earbud 50 includes a flat surface region 52 (hereinafter flat face 52) which is shaped as an ellipse with an area of about 35 mm² and a length of about 10 mm along its long axis.

The housing surfaces surrounding flat face 52 are shaped to create a relatively sharp angle at the transition with flat face 52 (maximal angle of 140 degrees). Flat face 52 is configured to anatomically match the relatively flat surface of the Tragus facing the ear canal, the point of optical coupling with sensor 22.

Thus, when eartip 18 of earbud 50 is inserted into the ear canal (FIG. 5b), the flat surface of flat face 52 and that of the tragus lock earbud 50 against rotation and stabilize sensor 22 against the tragus surface; further stabilization can be provided by stabilizing element 24. In addition, the sharp corners surrounding flat face 52 ensure that the user does not place the earbud in the wrong rotational angle about the nozzle, since any such positioning would lead to discomfort.

As used herein the term “about” refers to ±10%.

Additional objects, advantages, and novel features of the present invention will become apparent to one ordinarily skilled in the art upon examination of the following examples, which are not intended to be limiting.

### Examples

Reference is now made to the following example, which together with the above descriptions, illustrate the invention in a non limiting fashion.

An earbud including the present stabilizing element (shark fin-shaped) was tested against an identical earbud including a prior art stabilizing element (shark fin shaped). Both earbuds included a PPG-based sensor which was used to obtain heart rate from a tragus region of an ear. The two earbuds were tested with the same group of 15 subjects (mean ages 29±6, 7 male, 8 female); the subjects ran on a treadmill at a pace of 10 kilometers per hour for 8 minutes. Reference was a Polar H7 heart rate chest strap. The subjects were provided with the earbud and were given no instructions regarding in-ear placement. The position of the sensor on each earbud was tested based on signal to noise ratio (Table 2) and the performance of each earbud was tested relative to the readings from the chest strap (Table 1). The probability shown is for a single tailed paired T-test.

TABLE 1
performance tests of two stabilizing elements

| Error relative to chest strap | <5 bpm | <10 bpm | <15 bpm |
|---|---|---|---|
| reference (absolute value) | | | |
| Percent of time - Prior art fin | 86.5 ± 6.0 | 91.0 ± 4.1 | 95.8 ± 1.8 |
| Percent of time - Present fin | 91.5 ± 2.7 | 95.6 ± 1.6 | 97.5 ± 1.72 |
| P - T-Test | <0.015 | <0.01 | <0.02 |

TABLE 2
Sensor Placement Test

| Fin | % Subjects above SNR threshold |
|---|---|
| Prior Art | 73.3% |
| New Fin | 93.3% |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. An earbud comprising:
(a) a housing enclosing a speaker;
(b) an optical sensor positioned on said housing such that said optical sensor contacts a surface of tragus when the earbud is positioned in an ear with said speaker directed into an ear canal; and
(c) a stabilizing element attachable to said housing and being configured for contacting a posterior wall of a cavum or incisura of said ear when the earbud is positioned in said ear with said speaker directed into said ear canal.

2. The earbud of claim 1, wherein said stabilizing element is attachable to said housing at one of several preset rotational positions around said housing.

3. The earbud of claim 1, wherein said optical sensor is a photoplethysmography (PPG) sensor.

4. The earbud of claim 1, further comprising an accelerometer positioned in or on said housing.

5. The earbud of claim 2, wherein said housing includes a plurality of circumferentially spaced engagement elements for engaging said stabilizing element at one of said several preset rotational positions around said housing.

6. The earbud of claim 1, wherein said stabilizing element is shaped as a shark fin with a base of said shark fin surrounding a portion of a circumference of said housing.

7. The earbud of claim 6, wherein a portion of a front curve extending from a base of said shark fin is configured for contacting said posterior wall of a cavum or incisura of said ear when the earbud is positioned in said ear with said speaker directed into said ear canal.

8. The earbud of claim 7, wherein said front curve has a radius of curvature of 12-15 mm.

9. The earbud of claim 6, wherein a tip of said shark fin contacts the superior Cymba or Antihelix region of said ear when the earbud is positioned in said ear with said speaker directed into said ear canal.

10-12. (canceled)

13. An earbud comprising:
(a) a housing enclosing a speaker, said housing having a flat face being for contacting a surface of a tragus when the ear bud is positioned in an ear with said speaker directed into an ear canal at a specific rotational angle with respect to an axis extending out of said ear canal; and
(b) an optical sensor positioned on said flat face of said housing.

14. The earbud of claim 13, further comprising a stabilizing element attachable to said housing and being configured for contacting a posterior wall of a cavum or incisura of said ear when the earbud is positioned in said ear with said speaker directed into said ear canal.

15. The earbud of claim 14, wherein said stabilizing element is attachable to said housing at one of several preset rotational positions around said housing.

16. The earbud of claim 13, wherein said optical sensor is a photoplethysmography (PPG) sensor.

17. The earbud of claim 13, further comprising an accelerometer positioned in or on said housing.

18. The earbud of claim 13, further comprising a digital signal processor (DSP) within said housing.

19. The earbud of claim 14, wherein said stabilizing element is shaped as a shark fin with a base of said shark fin surrounding a portion of a circumference of said housing.

20. The earbud of claim 19, wherein a portion of a front curve extending from a base of said shark fin is configured for contacting said posterior wall of a cavum or incisura of said ear when the earbud is positioned in said ear with said speaker directed into said ear canal.

21. The earbud of claim 20, wherein said front curve has a radius of curvature of 12-15 mm.

22. The earbud of claim 19, wherein a tip of said shark fin contacts the superior Cymba or Antihelix region of said ear when the earbud is positioned in said ear with said speaker directed into said ear canal.
